(19) United States
(12) Patent Application Publication
(10) Pub. No.: US 2011/0046534 A1
(43) Pub. Date: Feb. 24, 2011

(54) DEVICE AND METHOD FOR MONITORING AN ACCESS TO A PATIENT, IN PARTICULAR A VASCULAR ACCESS DURING AN EXTRACORPOREAL BLOOD TREATMENT

(76) Inventor: Malte Gross, Niederwerrn (DE)

Correspondence Address: Kenyon & Kenyon LLP, One Broadway, New York, NY, 10004, US

(21) Appl. No.: 12/920,636

(22) PCT Filed: Mar. 3, 2009
(86) PCT No.: PCT/EP2009/001473

§ 371 Date: Oct. 20, 2010

(30) Foreign Application Priority Data
Mar. 7, 2008 (DE) 10 2008 013 090.7

(51) Int. Cl. A61M 1/00; A61B 5/05

(52) U.S. Cl. 604/5.01; 600/407

(57) ABSTRACT

The invention relates to the monitoring of an access to a patient at a puncture point, in particular the monitoring of the arterial and/or venous vascular access during an extracorporeal blood treatment. The device according to the invention is a magnetoelastic moisture sensor, which is embedded in an absorbent material and is laid on the puncture point. For the case in which infusion solution and/or blood exits from the puncture point, the incorrect seating of the puncture cannula can be recognized reliably using the device according to the invention. The absorbent material having the embedded magnetoelastic moisture sensor can be laid like conventional bandage material or a gauze bandage on the puncture point.

### CROSS REFERENCE TO RELATED APPLICATIONS

This is a 371 national phase application of PCT/EP2009/001473 filed Mar. 3, 2009, claiming priority to German Patent Application No. 10 2008 013 090.7 filed Mar. 7, 2008.

### FIELD OF INVENTION

The present invention relates to a device for the detection of fluid for a device for monitoring an access to a patient, in particular for monitoring the arterial and/or venous vascular access in an extracorporeal blood treatment apparatus. Moreover, the present invention relates to a device for monitoring an access to a patient, in particular for monitoring a vascular access in an extracorporeal blood treatment, whereby the monitoring device comprises a device for the detection of blood. Furthermore, the present invention relates to an extracorporeal blood treatment apparatus with a device for monitoring the arterial and/or venous vascular access as well as a method for monitoring an access to a patient, in particular for monitoring the arterial and/or venous vascular access in an extracorporeal blood treatment.

In the field of medical technology, apparatuses are known with which fluids can be withdrawn from patients via a tube line or fluids can be fed to patients. The access to the patient generally takes place with a catheter for introduction into body organs or a cannula or needle for the puncturing of vessels. During examination or treatment, a proper access to the patient must be ensured. It is therefore necessary to monitor the patient access.

A case of application with particularly high demands on the reliability of the monitoring of the vascular access is extracorporeal blood treatment, in which blood is carried away from the patient via an arterial tube line which has an arterial puncture cannula (needle), the blood is passed through a dialyser and is fed back again to the patient via a venous blood line, which has a venous puncture cannula (needle). Despite regular monitoring of the patient access by hospital staff, there is in principle the risk of a puncture cannula slipping out of the patient's blood vessel unnoticed or even of both puncture cannulas slipping out of the patient's blood vessel. Whereas the slipping-out of the arterial cannula is associated with the sucking-in of air into the arterial tube line, which leads to an acoustic and/or optical alarm and to interruption of the treatment on account of air being detected on the machine side, the slipping-out of the venous cannula, and the free flow of blood into the surroundings feared as a result, cannot be readily detected. If the slipping-out of the venous cannula is not detected immediately, however, the patient can bleed to death.

### BACKGROUND OF THE INVENTION

Devices are known in the prior art which monitor the position of the arterial and/or venous cannula to detect a defective vascular access, in order to be able to detect the slipping-out of the cannula from the vessel.

U.S. Pat. No. 5,578,003 describes a safety device for a tube line conveying blood, wound discharge or infusion, said safety device reacting to a relative change in position of the tube line.

There is known from German Patent No. DE 199 53 068 A1 a mechanical safety device, which can be fixed to the blood line of a dialysis machine. The known safety device comprises elastically pretensioned clamping jaws, which are held in the opened position by a locking bar fixed to the patient's body. The locking bar is torn away in the event of a change in position of the blood line, so that the clamping jaws pinch off the tube.

Furthermore, devices for monitoring a vascular access are known, which are based on monitoring the venous and arterial pressure in the extracorporeal blood circuit. WIPO Patent Publication No. WO 97/10013, for example, describes a monitoring device based on monitoring the pressure in the extracorporeal circuit. An advantage of these monitoring devices lies in the fact that a sensor on the patient side is not required. However, there is the risk that only the complete slipping-out of the cannula may be detected, but not seeping bleeding due to a partial dislocation of the cannula. Incompletely closed Luer-Lock connectors can likewise not be detected with sufficient reliability.

Apart from the devices described above, monitoring devices are known which detect the blood issuing at the puncture point. WIPO Patent Publication No. WO 2006/008866 A1 and WIPO Patent Publication No. WO 99/24145 A1, for example, describe devices for monitoring an access to a patient which comprise a moisture-sensitive sensor which detects the blood issuing at the puncture point. A moisture sensor for detecting bleeding is also known from U.S. Pat. No. 6,445,304 B1.

US Patent Publication No. 2002/198483 A1 describes a moisture sensor for the detection of blood at a puncture point in an extracorporeal blood treatment. The sensor is a conductive sensor, which is connected electrically to a direct or alternating voltage source, whereby the voltage drop between the two conducting electrodes is measured. A capacitive sensor is proposed as an alternative to a conductive sensor. Furthermore, it is proposed, for reasons of sterility, to cover the puncture point with a sterile barrier, for example a sterile pad, onto which the conductive or capacitive moisture sensor is placed.

Magneto-elastic sensors are also known in the prior art for the detection of moisture or a fluid.

The article “Wireless Magnetoelastic Resonance Sensors: A Critical Review” from Sensors 2002, 2, 294-313, ISSN 1424-8220 describes a magneto-elastic sensor for the detection of moisture or a fluid. The article deals with the theoretical bases of such a sensor. The article describes how the resonance frequency of the magneto-elastic sensor diminishes with the increase in the moisture surrounding it. Moreover, apparatuses are described with which the change in the resonance frequency due to a change in the moisture can be measured.

The use of a magneto-elastic sensor in a hygiene product, in particular a nappy, is known from U.S. Pat. No. 7,176,344 B2. U.S. Pat. No. 7,176,344 B2, however, does not deal with the problem of monitoring a vascular access to a patient.

### SUMMARY OF THE INVENTION

The problem underlying the present invention is to monitor a vascular access to a patient, in particular an arterial and/or venous vascular access, in a straightforward manner with a high degree of reliability, whereby not only slipping-out, but also incorrect seating of the puncture cannula can be reliably detected.

The device according to the present invention for the detection of fluid for a device for monitoring an access to the patient is designed as a magneto-elastic moisture sensor, which is embedded in an absorbent material which is placed onto the puncture point.

In the event that infusion solution and/or blood issues from the puncture point and/or the puncture cannula, the incorrect seating of the puncture cannula can be reliably detected by means of the device according to the present invention.

The absorbent material with the embedded magneto-elastic moisture sensor can be placed onto the puncture point like a conventional dressing material or a gauze bandage. It is an advantage that, by using the device according to the present invention, the demands imposed by the Commission for Hospital Hygiene and Infection Prevention on hospital hygiene during dialysis are satisfied for practical purposes, since the puncture point is covered in a sterile manner by placing on the device according to the present invention. It is to be assumed that the absorbent material of the device according to the present invention is sterile.

The device according to the present invention can be made available at low cost in large piece numbers as an article intended for one-time use and can be handled like conventional dressing material for the avoidance of infections at puncture points. To that extent, the monitoring of the patient access does not require any additional measures.

The device according to the present invention for the detection of moisture preferably comprises means for fixing the absorbent material to the patient. The fixing means are preferably embodied as means with which the absorbent material can be fixed to the patient's skin in an adhesive manner. For this purpose, conventional plasters or the like, for example, can be used. The device according to the present invention, however, preferably comprises an adhesive strip or the like, which can be applied at the side of the absorbent material with which the device according to the present invention is placed at the puncture point on the patient's skin.

In an alternative embodiment, the pad has an annular or U-shaped form, so that the puncture point is surrounded partially or completely by the pad, but is not covered and thus remains visible. A sterile transparent covering can additionally be provided in order to cover the puncture point, which can be inspected. With the embodiment of a U-shaped pad, a magneto-elastic moisture sensor can be present in each case in one or in both legs. An annular pad can be embodied such that it is interrupted by a narrow slit at one point, so that the pad can be placed around the puncture point even after the puncture has already been made. An annular pad can be provided with one or more sensors.

The device according to the present invention for the detection of fluid is intended for a device for monitoring a patient access, in particular the arterial and/or venous vascular access in an extracorporeal blood treatment apparatus. The device for monitoring the vascular access, with which the device for the detection of fluid cooperates, comprises a measuring unit for measuring the resonance frequency of the magneto-elastic sensor and an evaluation unit for comparing the measured resonance frequency with a preset reference value.

In the case where the difference between the measured resonance frequency and the preset reference value is greater than a preset threshold value, the evaluation unit generates a control and/or alarm signal, which signals a defective patient access.

The device according to the present invention for monitoring the arterial and/or venous vascular access can form a separate module. The device according to the present invention, however, is preferably a component of the extracorporeal blood treatment apparatus.

If the device according to the present invention for monitoring the vascular access is a component of the extracorporeal blood treatment apparatus, the monitoring device can make use of the components that are already present in the blood treatment apparatus. These include, in particular, the central control and computing unit of the blood treatment apparatus, which comprises for example a microprocessor.

A preferred embodiment of the present invention makes provision such that the blood treatment is interrupted and/or an alarm is emitted when a control and/or alarm signal is generated. In order to interrupt the blood treatment, the blood pump, which is present in the blood treatment apparatus for conveying blood through the extracorporeal blood circuit, is preferably stopped. Moreover, the shut-off element, which is also disposed in the blood treatment apparatus in the extracorporeal blood circuit, is preferably closed. The arterial and/or the venous clamp of the extracorporeal blood circuit, for example, can be closed.

Various devices and methods are known in the prior art for the determination of the resonance frequency of the magneto-elastic sensor. In this regard, reference is made in particular to the article “Wireless Magnetoelastic Resonance Sensors: A Critical Review” from Sensors 2002, 2, 294-313, ISSN 1424-8220.

In a preferred embodiment of the present invention, an alternating magnetic field is generated at the puncture point by means of an exciter coil, the response of the magneto-elastic sensor to the alternating magnetic field being received by a detector coil. Since the signal transmission for the evaluation takes place wirelessly, lines can be dispensed with that would otherwise have to be provided inside the absorbent material. The production of the unit comprising the magneto-elastic moisture sensor and the absorbent material, which can also be designed as a pad or the like, is thus greatly simplified.

### BRIEF DESCRIPTION OF THE DRAWINGS

An example of embodiment of the present invention will be explained in greater detail below by reference to the drawings.

In the figures:

FIG. 1 shows the main components of a hemodialysis apparatus together with the device according to the present invention for monitoring the patient access, in a greatly simplified schematic representation.

FIG. 2 shows a section through the device according to the present invention for the detection of fluid for the device according to the present invention for monitoring the patient access.

FIG. 3 shows a view from beneath of the device according to the present invention for the detection of fluid.

FIG. 4 shows the main components of the device according to the present invention for monitoring the patient access.

FIG. 5 shows the device according to the present invention for the detection of fluid, said device lying on a puncture point.

### DETAILED DESCRIPTION OF THE DRAWINGS

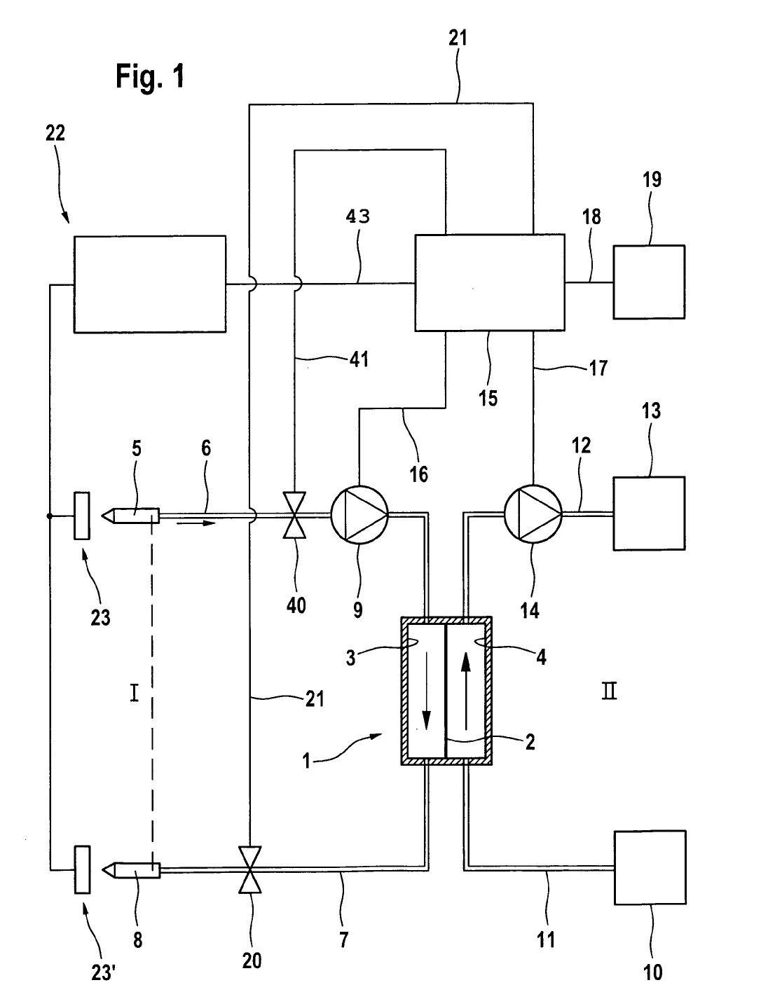

FIG. 1 shows the main components of a hemodialysis apparatus, which comprises a device for monitoring the venous and arterial vascular access. The hemodialysis apparatus comprises a dialyser 1, which is divided by a semipermeable membrane 2 into a blood chamber 3 and a dialysing fluid chamber 4. An arterial tube line 6 is connected by means of an arterial puncture cannula 5 to one of the patient's arteries, said arterial tube line leading to the inlet of chamber 3 of the dialyser. Leading away from the outlet of chamber 3 of the dialyser is a venous tube line 7, which is connected by means of a venous puncture cannula 8 to one of the patient's veins. Arterial tube line 6 is inserted into an occluding blood pump 9, which conveys the blood in extracorporeal blood circuit I.

Dialysing fluid circuit II of the hemodialysis apparatus comprises a dialysing fluid source 10, to which a dialysing fluid supply line 11 is connected, which leads to the inlet of dialysing fluid chamber 4 of the dialyser. Departing from the outlet of dialysing fluid chamber 4 of dialyser 1 is a dialysing fluid discharge line 12, which leads to a drain 13. A dialysing fluid pump 14 is incorporated into dialysing fluid discharge line 12.

The control of the dialysis apparatus is assumed by a central control unit 15, which controls blood pump and dialysing fluid pump 9, 14 via control lines 16, 17. Central control unit 15 is connected via a data line 18 to an alarm unit 19, which emits an optical and/or acoustic alarm in the event of a malfunction.

Located downstream of blood chamber 3 of the dialyser on venous tube line 7 is an electro-magnetically actuated venous tube clamp 20, which is closed via a further control line 21 by central control unit 15 when a defective vascular access is ascertained, for example after the complete slipping-out of the cannula from the vessel or a partial dislocation of the cannula which leads to the issuing of blood. An electro-magnetically actuated arterial tube clamp 40, which is closed via a further control line 41 by central control unit 15 when a defective vascular access is ascertained, is located upstream of blood chamber 3 of the dialyser on arterial tube line 6.

A device 22, which will also be described in detail below, is provided for monitoring the vascular access. Device 22 for monitoring the vascular access can be a component of the blood treatment apparatus or form a separate module. In the present example of embodiment, monitoring device 22 is a component of the dialysis apparatus, inasmuch as the monitoring device makes use of various components which are in any case present in the dialysis apparatus, for example parts of central control unit 15 (microprocessor) or alarm unit 19.

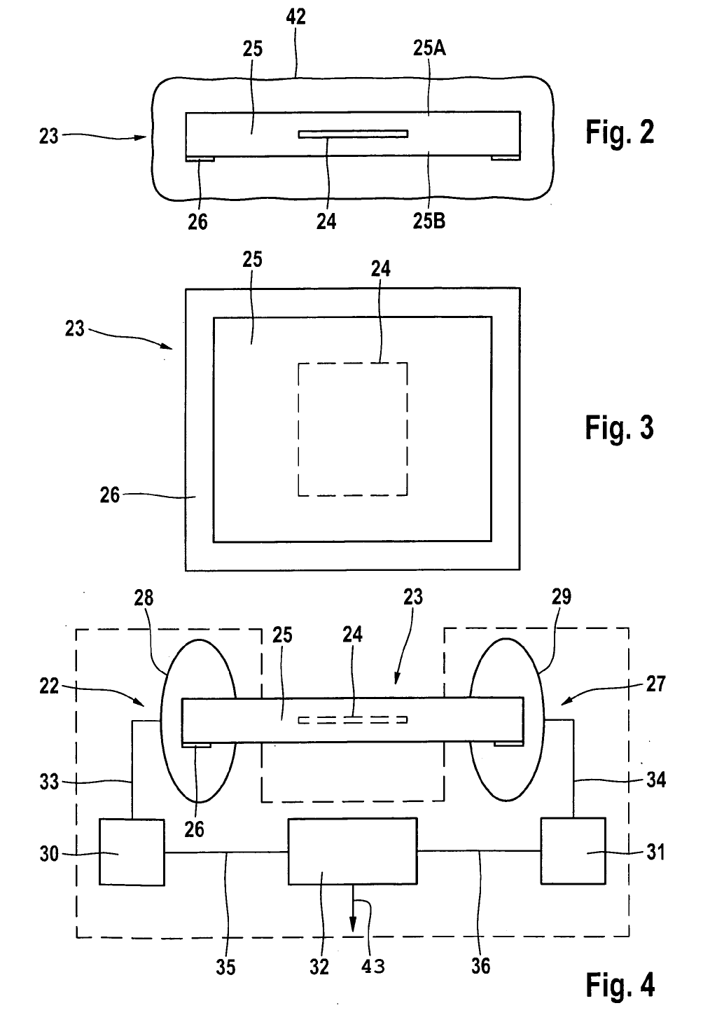

Device 22 for monitoring the vascular access comprises a device 23 for the detection of fluid, said device being placed onto the puncture point. Device 23 for the detection of fluid will be described in detail below by reference to FIGS. 2 and 3.

Device 23 for the detection of fluid comprises a magneto-elastic moisture sensor 24, which is designed as a flexible flat strip.

Since a magneto-elastic moisture sensor belongs as such to the prior art, the moisture sensor will not be described in detail. Such a moisture sensor is described for example in the article “Wireless Magnetoelastic Resonance Sensors: A Critical Review” from Sensors 2002, 2, 294-313, ISSN 1424-8220, to which reference is expressly made for the purposes of the disclosure.

Magneto-elastic moisture sensor 24 is completely embedded in an absorbent material 25. Absorbent material 25 comprises an essentially rectangular upper layer 25A and an essentially rectangular lower layer 25B with identical dimensions. Moisture sensor 24 is completely enclosed between upper and lower layer 25A, 25B, which form a unit. FIGS. 2 and 3 show a section through an absorbent material with the moisture sensor as well as a view from below of the absorbent material with the sensor.

At the underside, the absorbent material is provided with a peripheral adhesive strip 26, with which the absorbent material with the moisture sensor, which is also referred to below as a pad, is stuck firmly to the patient's skin at the puncture point. Adhesive strip 26 should adhere firmly to the patient's skin, but should be able to be easily pulled off. Instead of a peripheral adhesive strip, a plurality of adhesive strips can also be provided.

The pad represents an article intended for one-time use, which can be made available packaged in a sterile manner in a film packing 42.

FIG. 4 shows the main components of the device for monitoring the vascular access. The device for monitoring vascular access 22 comprises a measuring unit 27 for measuring the resonance frequency of magneto-elastic moisture sensor 24, which is embedded in sterile absorbent material 25.

Frequency f of the fundamental oscillation of the magneto-elastic sensor in the form of a parallelepiped-shaped strip of length L amounts to:

$$f = \frac{1}{2 L} \sqrt{\frac{{E}_{S}}{{ρ}_{S} ( 1 - {σ}^{2} )}} \qquad ( 1 )$$

Here, E_{S} is the modulus of elasticity, ρₛ the density and σ Poisson's ratio (transverse contraction ratio) of the strip material. An additional mass coverage Δm of the sensor can be described by a change in density ρₛ and leads approximately to the following change in frequency,

$$Δ f = - {f}_{0} \frac{Δ m}{2 {m}_{S}} \qquad ( 2 )$$

Measuring unit 27 for detecting the change in frequency comprises an exciter coil 28 and a detector coil 29 as well as a function generator 30, a lock-in-amplifier 31 and an evaluation unit 32. Via electrical connection lines 33 and 34 as well as 35 and 36, which are represented solely in outline in FIG. 4, function generator 30 is connected to exciter coil 28, lock-in-amplifier 31 to detector coil 29 and evaluation unit 32 on the one hand to function generator 30 and on the other hand to the lock-in-amplifier. Evaluation unit 32 can be a conventional computer (microprocessor), which can also be a component of central control unit 15 of the dialysis apparatus.

Function generator 30 generates an alternating voltage with a constant amplitude and frequency, as a result of which the exciter coil generates an alternating magnetic field which penetrates moisture sensor 24. Detector coil 29 measures the response of the moisture sensor to the alternating magnetic field. The frequency of the alternating voltage generated by the function generator is changed, the response of the moisture sensor being measured and amplified with the exciter coil and the lock-in-amplifier. While the frequency of the alternating voltage signal is changed, evaluation unit 32 determines the frequency at which the amplitude of the signal detected by detector coil 29 is greatest. This frequency corresponds to the resonance frequency of moisture sensor 24.

The measured resonance frequency of moisture sensor 24 is compared in evaluation unit 32 with a preset reference value. If the amount of the difference between the measured resonance frequency and the preset reference value is greater than a preset threshold value, evaluation unit 32 generates a control and/or alarm signal. The reference value can be calculated in a known manner from the given sensor data according to equation (1). Production-related variations in the sensor properties are not however taken into account here. It may therefore be advisable to measure the reference value by means of a sample measurement, e.g. during the commissioning of the sensor, and to store the measured value as a reference value.

The detection of blood at the puncture point is based on the fact that the resonance frequency of magneto-elastic moisture sensor 24 changes in the presence of wetting of magneto-elastic moisture sensor 24 with moisture (fluid). The resonance frequency falls with increasing moisture. If blood issues at the puncture point, the difference between the measured resonance frequency and the preset reference value becomes greater than a preset threshold value. The preset reference value for the reference frequency is a value which is assumed when the moisture sensor is dry or is exposed to only a small amount of moisture.

Since the moisture sensor is embedded in the absorbent material, the effect of the change in the frequency due to the change in the moisture is increased considerably, so that the signal evaluation is even easier and more reliable.

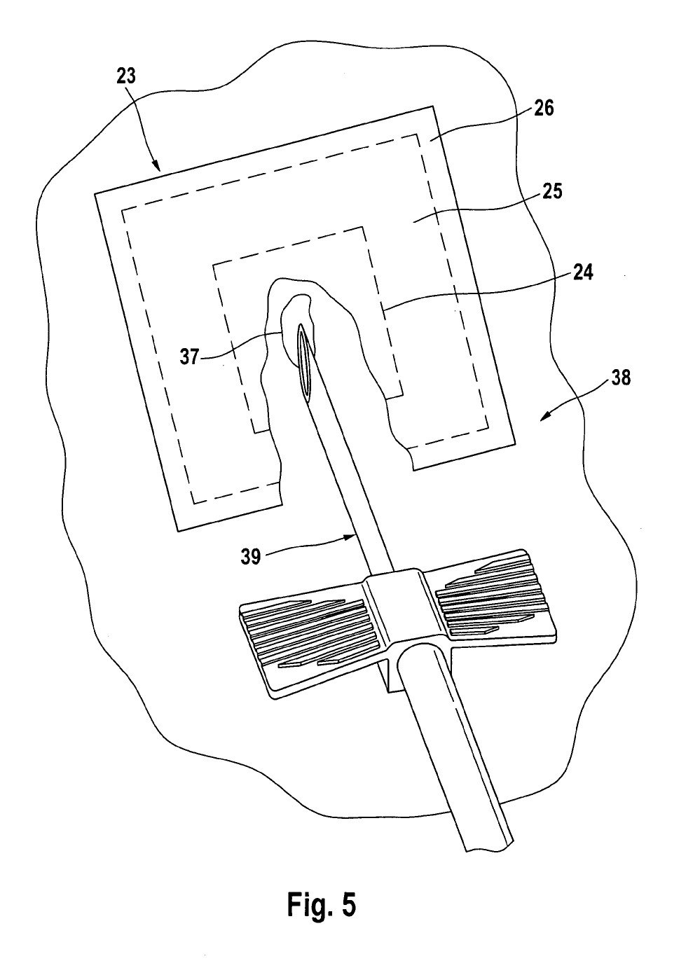

FIG. 5 shows the area of puncture point 37 at which the patient's skin 38 is perforated with a puncture cannula 39. Pad 23 is placed onto puncture point 37, so that the puncture point is covered in a sterile manner. Pad 23 adheres by means of adhesive strip 26 to the patient's skin. After the blood treatment, the pad is removed again and the cannula withdrawn from the vessel.

Evaluation unit 32 of device 22 for monitoring the vascular access is connected via line 43 to central control unit 15 of the dialysis apparatus. When the monitoring device detects a defective vascular access, i.e. the issuing of blood from the puncture point, central control unit 15 of the dialysis apparatus receives the control and/or alarm signal of the monitoring device. Control unit 15 then closes electro-magnetically actuated shut-off element 20 in venous tube line 7 and stops blood pump 9 in arterial tube line 6. The extracorporeal treatment is thus interrupted, so that a further free flow of blood from the venous needle is not possible.

FIG. 1 shows an example of embodiment in which monitoring device 22, represented solely in outline, comprises a venous and arterial device 23, 23′ for the detection of blood, in order to be able to monitor both the venous and the arterial vascular access.

On account of the dependence of the resonance frequency on the viscosity of the fluid, a distinction can be made between different fluids, for example between blood issuing at the puncture point or an increase in moisture caused by perspiration. The possibility of a false alarm can thus be eliminated by targeted fixing of the threshold value.

In a preferred embodiment, moisture sensor 24 is coated. A coating can be provided which reacts with blood, so that the properties of the blood change. A coating is preferably provided which coagulates blood. The coating can contain an enzyme which coagulates blood. An acid or a base can however also be contained in the coating, which denatures the blood protein, such as for example albumin.

For the further reliable distinction between the issuing of blood and an increase in moisture that is not due to the issuing of blood, two or more than two magneto-elastic moisture sensors can also be embedded in absorbent material 25 of pad 23 in a further preferred embodiment, said moisture sensors differing in their electrical properties, especially the dependence of the resonance frequency on moisture.

For example, two magneto-elastic sensors with different surface roughness can be provided in order to determine the density and viscosity of the fluid from the difference in the respective resonance frequencies of the two sensors in order to distinguish between different events.

In a preferred embodiment, exciter coil 28 and detector coil 29 are integrated in a dialysis bed or in a dialysis chair. Instead of separate coils 28 and 29, however, a combined exciter and detector coil can also be provided, which can be integrated into the bed or the chair. In the case of a dialysis bed, exciter coil 28 and detector coil 29 or the combined exciter and detector coil are preferably integrated into the lying area of the dialysis bed. In the case of a dialysis chair, the exciter coil and the detector coil or the combined exciter and detector coil are preferably integrated into the armrest.

In a further embodiment, the function of the exciter coil and the detector coil is achieved with only one coil which performs the function of both coils.

The electrical connection between exciter coil 28 and detector coil 29 on the one hand and the blood treatment machine on the other hand can take place by cable. It is however also possible to provide a wireless connection, e.g. a radio connection, between the coils on the one hand and the blood treatment machine on the other hand. The unit of exciter coil and detector coil preferably has its own power supply, e.g. a battery, so that electrical lines to a main supply can be dispensed with.

1-15. (canceled)

16. A detection device for the detection of fluid, said detection device configured for use with a monitoring device for monitoring an access to a patient whereby fluid is provided to a patient or carried away from the patient via a tube line, the detection device comprising:
a magneto-elastic moisture sensor embedded in an absorbent material.

17. The detecting device according to claim 16, wherein the device for the detection of fluid further comprises:
a means for fixing the absorbent material to the patient.

18. The detecting device according to claim 17, wherein the means for fixing are designed such that the absorbent material can be fixed to the patient's skin in an adhesive manner.

19. A monitoring device for monitoring an access to a patient comprising:
the detection device of claim 16;
a measuring unit for measuring the resonance frequency of the magneto-elastic moisture sensor; and
an evaluation unit for comparing the measured resonance frequency with a preset reference value, the evaluation unit configured to generate a control or alarm signal, which signals a defective patient access, if the amount of the difference between the measured resonance frequency and the preset reference value is greater than a preset threshold value.

20. The monitoring device according to claim 19, wherein the measuring unit for measuring the resonance frequency comprises:
an exciter coil for generating an alternating magnetic field; and
a detector coil for detecting an alternating magnetic field.

21. A blood treatment apparatus with an extracorporeal blood circuit, comprising:
an arterial tube line with an arterial puncture cannula;
a venous tube line with a venous puncture cannula; and
the monitoring device for monitoring the arterial or venous vascular access of claim 32.

22. The blood treatment apparatus according to claim 21, wherein the blood treatment apparatus further comprises:
a control unit that cooperates with the monitoring device in such a way that the blood treatment is interrupted or an alarm is emitted when the control or alarm signal is generated.

23. The blood treatment apparatus according to claim 22, wherein the blood treatment apparatus further comprises:
a blood pump for conveying blood through the extracorporeal blood circuit,
wherein the control unit cooperates with the monitoring device in such a way that the blood pump is stopped when the evaluation unit generates the control or alarm signal.

24. The blood treatment apparatus according to claim 22, wherein the blood treatment apparatus further comprises:
a shut-off element disposed in the extracorporeal blood circuit,
wherein the control unit cooperates with the monitoring device in such a way that the shut-off element is closed when the evaluation unit generates the control or alarm signal.

25. The blood treatment apparatus according to claim 22, wherein the blood treatment apparatus further comprises:
an alarm unit which emits an acoustic and/or optical alarm,
wherein the control unit cooperates with the monitoring device in such a way that the alarm unit emits an alarm when the evaluation unit generates the control or alarm signal.

26. A method of detecting blood at a puncture point of a patient comprising:
placing at least one magneto-elastic moisture sensor embedded in an absorbent material at the puncture point.

27. A method for monitoring an access to a patient, whereby fluid is provided to a patient or carried away from the patient via a tube line, comprising:
placing a magneto-elastic sensor embedded in absorbent material onto a puncture point on the patient,
determining the resonance frequency of the magneto-elastic moisture sensor;
determining if there is a defective vascular access on the basis of the change in the resonance frequency; and
emitting an alarm or interrupting the withdrawal or the supply of fluid in the event of a defective vascular access being ascertained.

28. The method according to claim 27 for monitoring an access to a patient, wherein the access is an arterial or venous vascular access in an extracorporeal blood treatment apparatus, said method further comprising:
providing a patient's blood to the patient via a venous tube line that has a venous puncture cannula;
carrying away blood from the patient via an arterial tube line that has an arterial puncture cannula; and
interrupting the blood treatment or emitting an alarm in the event of a defective vascular access being ascertained.

29. The method according to claim 28, wherein the step of interrupting the blood treatment comprises:
stopping a blood pump disposed in an extracorporeal blood circuit or closing a shut-off element disposed in the extracorporeal blood circuit.

30. The method according to claim 28, wherein determining the resonance frequency of the magneto-elastic sensor comprises:
generating an alternating magnetic field at a puncture point by an exciter coil; and
receiving the response of the magneto-elastic sensor to the alternating magnetic field by a detector coil.

31. The detection device according to claim 16, wherein the monitoring device is for monitoring an arterial access or a venous vascular access in an extracorporeal blood treatment apparatus, whereby a patient's blood is provided to the patient via a venous tube line that has a venous puncture cannula, and blood is carried away from the patient via an arterial tube line that has an arterial puncture cannula.

32. The monitoring device according to claim 19, wherein the monitoring device is for monitoring an arterial access or a venous vascular access in an extracorporeal blood treatment apparatus, whereby a patient's blood is provided to the patient via a venous tube line that has a venous puncture cannula, and blood is carried away from the patient via an arterial tube line that has an arterial puncture cannula.

33. The method according to claim 26, for monitoring an arterial or venous vascular access in an extracorporeal blood treatment apparatus, further comprising:
providing a patient's blood is to the patient via a venous tube line that has a venous puncture cannula; and
carrying blood away from the patient via an arterial tube line that has an arterial puncture cannula.

34. A monitoring device for monitoring an access to a patient comprising:
the detection device of claim 17;
a measuring unit for measuring the resonance frequency of the magneto-elastic moisture sensor; and
an evaluation unit for comparing the measured resonance frequency with a preset reference value, the evaluation unit configured to generate a control or alarm signal, which signals a defective patient access, if the amount of the difference between the measured resonance frequency and the preset reference value is greater than a preset threshold value.

35. The blood treatment apparatus according to claim 21, wherein the measuring unit for measuring the resonance frequency comprises:
an exciter coil for generating an alternating magnetic field; and
a detector coil for detecting an alternating magnetic field.
